(19) United States
(12) Patent Application Publication
(10) Pub. No.: US 2026/0346012 A1
(43) Pub. Date: Nov. 5, 2026

(54) PAIN MANAGEMENT VIA VENTRAL SPINAL CORD STIMULATION

(71) Applicant: Case Western Reserve University, Cleveland, OH (US)

(72) Inventors: Gustaf Van ACKER, Cleveland, OH (US); Chong KIM, Cleveland, OH (US)

(21) Appl. No.: 19/120,301

(22) PCT Filed: Oct. 13, 2023
(86) PCT No.: PCT/US2023/076762

§ 371 Date: Apr. 11, 2025

Related U.S. Application Data
(60) Provisional application No. 63/415,734, filed on Oct. 13, 2022.

(51) Int. Cl. A61N 1/36; A61N 1/05

(52) U.S. Cl. CPC A61N 1/36062; A61N 1/0553; A61N 1/36071; A61N 1/36139; A61N 1/36171

(57) ABSTRACT

Ventral spinal cord stimulation directly modulates neural tracts that include fibers carrying pain signals. At least one electrode is implanted within a ventral space of a spinal column of a subject and is positioned near an anterior and/or a lateral spinothalamic tract, which can carry pain signals. A signal generator generates an electrical signal configured to stimulate at least one sensory fiber carrying a pain signal in the anterior and/or the lateral spinothalamic tract and sends the electrical signal to the at least one electrode. The electrical signal is then applied by the at least one electrode to the anterior and/or the lateral spinothalamic tract to stimulate the at least one sensory fiber to override the pain signal with non-painful stimulus induced activity to at least partially relive, control, or alleviate pain in the subject.

### RELATED APPLICATIONS

This application claims priority to U.S. Provisional Application Ser. No. 63/415,734, filed Oct. 13, 2022, entitled “VENTRAL AND VENTROLATERAL SPINAL CORD STIMULATION FOR CHRONIC PAIN”. The entirety of this application is hereby incorporated by reference for all purposes.

### TECHNICAL FIELD

The present disclosure relates to pain management, and more specifically, to systems and methods for pain management via ventral spinal cord stimulation.

### BACKGROUND

Chronic pain is both disruptive and difficult to manage. Spinal cord stimulation (SCS) has emerged as a non-pharmaceutical treatment option for chronic pain. The posterior elements of the dorsal column of the spinal cord are traditionally and currently the anatomical target of choice for stimulation to treat a majority of chronic pain conditions. Stimulating the posterior elements of the dorsal column (via dorsal SCS) relies on indirect modulation of tracts transmitting a pain signal in the spinal cord through intraspinal connectivity. However, for a significant number of patients, stimulating the posterior elements of the dorsal column (via dorsal SCS) to manage chronic pain has had limited or no success in managing pain.

### SUMMARY

Provided herein is a pain treatment procedure that delivers stimulation more directly to the anterior and/or lateral spinothalamic tracts on the ventral side of spinal cord (via ventral column spinal cord stimulation (SCS)), which include the fibers that transmit pain signals through the spinal cord. The procedure utilizes electrical stimulation through the ventral side of spinal cord (ventral SCS) rather than indirect electrical stimulation through the dorsal column (dorsal SCS) to provide relief for chronic pain (as well as other types of pain).

In one aspect, the present disclosure includes a method for ventral spinal cord stimulation for pain management. The method includes: implanting at least one electrode within a ventral space of a spinal column of a subject, wherein the at least one electrode is positioned near an anterior and/or a lateral spinothalamic tract; generating, by a signal generator, an electrical signal configured to stimulate at least one sensory fiber carrying a pain signal in the anterior and/or the lateral spinothalamic tract; and applying the electrical signal via the at least one electrode to the anterior and/or the lateral spinothalamic tract to stimulate the at least one sensory fiber to override the pain signal with non-painful stimulus induced activity to at least partially relieve a pain felt by the subject.

In another aspect, the present disclosure includes a system for ventral spinal cord stimulation, which can include at least a signal generator and at least one electrode. The signal generator is configured to generate an electrical signal, wherein the electrical signal is configured to stimulate at least one sensory fiber carrying a pain signal in an anterior and/or a lateral spinothalamic tract. And at least one electrode is configured to be implanted within a ventral space of the spinal column of the subject near the anterior and/or the lateral spinothalamic tract. The at least one electrode applies the electrical signal to the at least one sensory fiber to stimulate the at least one sensory fiber to override the pain signal with non-painful stimulus induced activity to at least partially relieve a pain felt by the subject.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 is a diagram showing a system that can be used to provide ventral spinal cord stimulation (SCS);

FIG. 2 shows an example placement of a stimulating electrode of the system of FIG. 1 with respect to the spinal cord;

FIG. 3 is a diagram showing another system that can be used for both dorsal and ventral SCS;

FIG. 4 shows an example process flow diagram of a method for pain management with ventral SCS;

FIG. 5 shows an example process flow diagram of a method for combining ventral and dorsal SCS;

FIG. 6 shows an example process flow diagram of a method for controlling ventral SCS with feedback;

FIG. 7 shows x-ray images of two dorsal SCS leads and one ventral SCS lead positioned relative to the spinal cord;

FIG. 8 shows x-ray images of the ventral SCS lead positioned relative to the spinal cord; and

FIG. 9 shows an example thoracic cross-section illustration of the placement of the two dorsal SCS leads and the ventral SCS lead relative to the spinal cord.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an,” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

It will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

As used herein, the term “pain management” refers to relieving and/or controlling chronic and/or acute pain of a patient with the goal of easing the patient's suffering and improving the patient's quality of life. Pain refers to any physical suffering or discomfort of the patient and can include one or more signals that can be transmitted through one or more sensory nerve fibers to the brain. Pain management can be multidisciplinary and can include pharmacological measures, physical measures, interventional procedures, physical therapy, psychological measures, or the like. Spinal cord stimulation (SCS) is one form of pain management (e.g., a physical measure), which can be combined with one or more other measures.

As used herein, the term “spinal cord stimulation”, also referred to as “SCS”. refers to a method for management of pain (generally chronic but may be acute) where at least one electrode is positioned in epidural space adjacent the spinal cord to apply an electrical signal (generated by a signal generator) to control and/or relieve feelings of pain. SCS may be dorsal SCS and/or ventral SCS.

As used herein, the term “dorsal” SCS refers to the traditional method and positioning for spinal cord stimulation where the at least one electrode is positioned on a dorsal side of the spinal cord to indirectly modulate sensory nerve fibers in the anterior and/or lateral spinothalamic tracts of the spinal cord through intraspinal connectivity. Dorsal SCS can be at least partially ineffective (e.g., no pain is controlled, pain control is minimal, or the like) in a significant number of patients.

As used herein, the term “ventral” SCS refers to an improved method and positioning of spinal cord stimulation where the at least one electrode is positioned in the ventral epidural space adjacent the spinal cord to directly modulate sensory nerve fibers in the anterior and/or lateral spinothalamic tracts of the spinal cord.

As used herein, the term “electrical signal” can refer to a function that conveys information an electric phenomenon (e.g., current, voltage, etc.) that varies with time and/or space to at least one neural fiber and can alter conduction in the at least one neural fiber to reduce a feeling of pain. For example, the electrical signal can be an alternating current (e.g., kilohertz frequency alternating current that varies in amplitude for at least a portion of time) signal and/or a direct current (e.g., constant amplitude for at least a portion of time) signal.

As used herein, the term “electrode” refers to a conductor through which electricity enters or leaves an object, substance, or region. For example, an electrode can be implanted on the ventrolateral aspect of the spinal cord. The electrode can be any known shape and/or configuration for spinal implantation. For example, an electrode can be a multi-contact lead with a cylindrical shape. In another example, an electrode can be one or more paddle shaped leads.

As used herein, the terms “subject” and “patient” can be used interchangeably and refer to any warm-blooded organism including, but not limited to, a human being, a pig, a rat, a mouse, a dog, a cat, a goat, a sheep, a horse, a monkey, an ape, a rabbit, a cow, etc.

As used herein, the term “medical professional” can refer to an individual who provides care to a patient. A medical professional can be, for example, a doctor, a physician's assistant, a student, a nurse, a caregiver, or the like.

### II. Overview

Chronic pain is often difficult to manage and can severely impact a patient's quality of life. Spinal cord stimulation (SCS) has emerged as a non-pharmaceutical treatment option for chronic pain management. Traditionally, the posterior elements of the dorsal column of the spinal cord have been the anatomical target of choice for stimulation to treat a majority of chronic pain conditions (e.g., complex regional pain syndrome, neuropathic pain, radicular pain, diabetic neuropathy, nonsurgical low back pain, failed back surgery syndrome, etc.). Stimulating the posterior elements of the dorsal column (via dorsal SCS) relies on indirect modulation of tracts transmitting a pain signal in the spinal cord through intraspinal connectivity. However, for a significant number of chronic pain patients, dorsal SCS has had limited or no success in managing the pain.

The anterior and lateral spinothalamic tracts carry pain signal from the periphery to the brain and, while not wishing to be bound by theory, it is believed that modulation of the anterior and lateral spinothalamic tracts may act through a hijacking mechanism, whereby stimulation eliminates and replaces the firing of pathological pain signals. Described herein are systems and methods for pain management with ventral spinal cord stimulation (ventral SCS). Ventral SCS applies electrical stimulation more directly to the anterior and lateral spinothalamic tracts of the spinal cord than dorsal SCS. By more directly stimulating nerve fibers that run through the anterior and lateral spinothalamic tracts ventral spinal cord stimulation can relieve pain at least as well, if not better, than traditional dorsal column stimulation.

### III. System

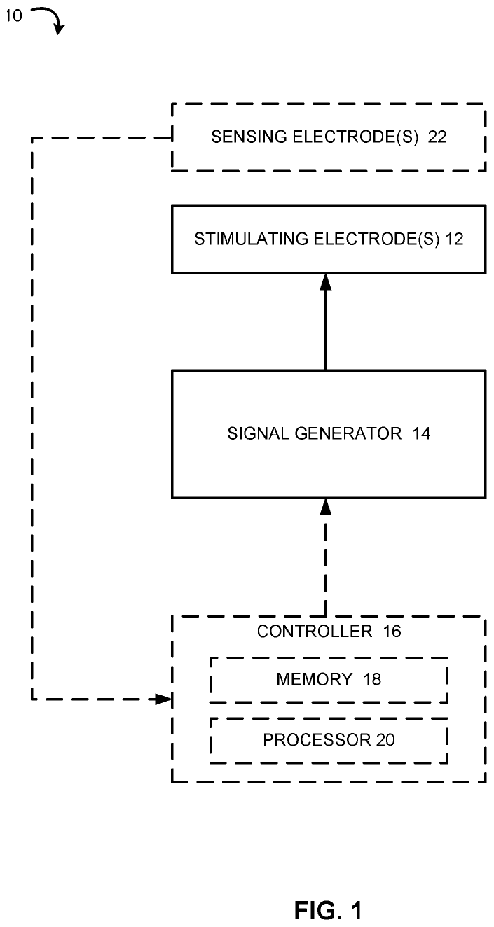

Provided herein is a system 10 (FIG. 1) for ventral Spinal Cord Stimulation (SCS). In a user suffering from pain (e.g., chronic pain and/or acute pain, referred to generally as “pain”), ventral SCS can be used as a treatment to manage the pain. Ventral SCS is at least equivalent to and may even be superior to traditional dorsal SCS. While not wishing to be bound by theory, it is postulated that ventral stimulation can directly modulate sensory fibers in the anterior and lateral spinothalamic tracts of the spinal cord (also referred to as “pain fibers”) to at least partially relieve and/or control pain felt by the patient undergoing the ventral SCS. As a non-limiting example, the pain treated and relieved by ventral SCS can be back pain, leg pain, neck pain, and/or neck pain. The pain can be caused, for example, by chronic pain syndrome, non-surgical back pain, complex regional pain syndrome, diabetic neuropathy, small fiber neuropathy, peripheral ischemia, surgery, or the like.

The system 10 can include at least one electrode (stimulating electrode(s) 12) electrically connected to a signal generator 14. The at least one electrode (one or more stimulating electrodes, represented by stimulating electrode(s) 12) can be implanted at a location in a ventral space (e.g., the ventral epidural space or on the ventral side of the spinal column) of the spinal column of a subject. It should be understood that the stimulating electrode 12 can be implanted at the location in the ventral space (e.g., any location in the ventral epidural space or on the ventral side of the spinal column with the ability to relieve pain upon stimulation) depending on the patient's ailment.

The one or more stimulating electrodes, represented by stimulating electrode(s) 12, can apply an electrical signal to at least one sensory fiber that can carry a pain signal in an anterior and/or a lateral spinothalamic tract of the spinal cord (also referred to as “pain fibers”). The signal generator 14 can generate the electrical signal to stimulate the at least one sensory fiber carrying the pain signal in the anterior spinothalamic tract and/or the lateral spinothalamic tract of the spinal cord. By applying the electrical signal, the system 10 can at least partially treat, control, or otherwise alleviate pain in the subject. For example, the electrical signal can stimulate that at least one sensory fiber to override the pain signal with non-painful stimulus induced activity.

The one or more stimulating electrodes, represented by stimulating electrode(s) 12, can include one or more electrical contacts. For example, as described in the Experimental section, the one or more stimulating electrodes, represented by stimulating electrode(s) 12, can be a lead having 8 electrical contacts. When the one or more electrical contacts is a plurality of electrical contacts, the electrical signal can be applied via one or more of the plurality of electrical contacts. As an example, in some instances, a process/procedure can be undertaken to find the best/most ideal one or more electrical contacts of the plurality of electrode contacts for the stimulating electrode(s) 12 to deliver the stimulation that relieves the greatest amount of pain for the patient, often by selecting one or more of the plurality of electrical contacts, and/or more than one electrode, to deliver the stimulus.

The one or more stimulating electrodes, represented by stimulating electrode(s) 12, can be, for example, at least one percutaneously implanted electrode (e.g., cylindrical electrode), at least one implantable paddle electrode, or the like. The one or more stimulating electrodes, represented by stimulating electrode(s) 12, can be at least partially made of and/or (at least substantially) coated with any known biologically safe, conductive material. As an example, the one or more stimulating electrodes, represented by stimulating electrode(s) 12, can be made of platinum-iridium. The one or more stimulating electrodes, represented by stimulating electrode(s) 12, can be implanted at any vertebrae or segment at any level of the spine (e.g., thoracic, lumber, sacral, cervical, etc.) dependent on the pain to be at least partially relieved. For example, the one or more stimulating electrodes, represented by stimulating electrode(s) 12, can be implanted on at least one thoracic level of the spine to at least partially relieve pain of the back and/or legs. In another example, the one or more stimulating electrodes, represented by stimulating electrode(s) 12. can be implanted on at least one cervical level of the spine to at least partially treat pain of the arms. In some instances, the one or more stimulating electrodes, represented by stimulating electrode(s) 12, can be implanted on a right side of the spine to relieve a pain on the right side of the body and/or on the left side of the spine to relieve a pain on the left side of the body depending on the source and/or location of the pain.

The one or more stimulating electrodes, represented by stimulating electrode(s) 12, can be connected to the signal generator 14 (which may be at least partially implanted and/or at least partially external) by a wired connection and/or a wireless connection. For example, the signal generator 14 can be an implanted signal generator (such as an implantable pulse generator) and/or an external signal generator. In some instances, the signal generator 14 can include a rechargeable power source (e.g., a battery). The signal generator 14 can generate the electrical signal with one or more parameters, which can be pre-programmed and/or selected by a controller 16. For example, the electrical signal can have a frequency between 0.001 MHz and 1 MHz and a current between 0.001 mA and 50 mA, between 0.001 mA and 20 mA, between 0.001 mA and 10 mA, or preferably between 0.001 mA and 5 mA. The electrical signal can be an alternating current or a direct current waveform, a monophasic or biphasic waveform. The electrical signal can be applied continuously or pulsed. For example, the electrical signal can have parameters of Fast-Acting Sub-perception Therapy stimulation (current: 1.2 mA, 230 μs, frequency: 90 Hz, and an active recharge phase). The electrical signal can be generated and applied for a time period ranging from 1 second to all day (e.g., 24 hours) to at least partially relieve the pain of felt by the patient. The application can be pre-set (e.g., by a medical professional) and/or delivered at the request of the patient (a maximum delivery might be set).

The system 10 can optionally include controller 16 connected to the signal generator 14 by a wired and/or wireless connection. The controller 16 can be, for example, external to the patient's body, such as a smart phone, a tablet, a computer, a dedicated external device, or the like. The controller 16 can include a non-transitory memory 18 configured to store instructions and a processor 20 configured to execute the instructions. The instructions can include programing for the one or more parameters of the electrical signal into the signal generator 14. The one or more parameters can be pre-loaded in the memory 16, selected based on values and/or ranges in a look up table stored in the memory in response to a circumstance or manual input, and/or based on manual input (e.g., from a user interface (not shown) associated with the controller 14). The system can also include one or more sensing electrode(s) 22 in electrical communication with at least the controller 16. The one or more sensing electrode(s) 22 can record one or more evoked compound action potentials related to the pain of the subject. In one instance, the controller 16 can be a closed loop controller and can at least receive a recording of the one or more evoked compound action potentials from the one or more sensing electrode(s) 22, modulate at least one parameter of the electrical signal in response to the one or more evoked compound action potentials to improve pain management, and send the modulated at least one parameter to the signal generator 14 to update the electrical signal. Additionally or alternatively, the one or more sensing electrode(s) 22 can include or in addition to sensors that are not strictly electrodes, such as sensors that can detect muscle contraction or movements that may be painful, or pain related. In some instances, the presence of one or more sensing electrode(s) 22 and the controller 16 can make the system 10 fully and/or at least partially automated.

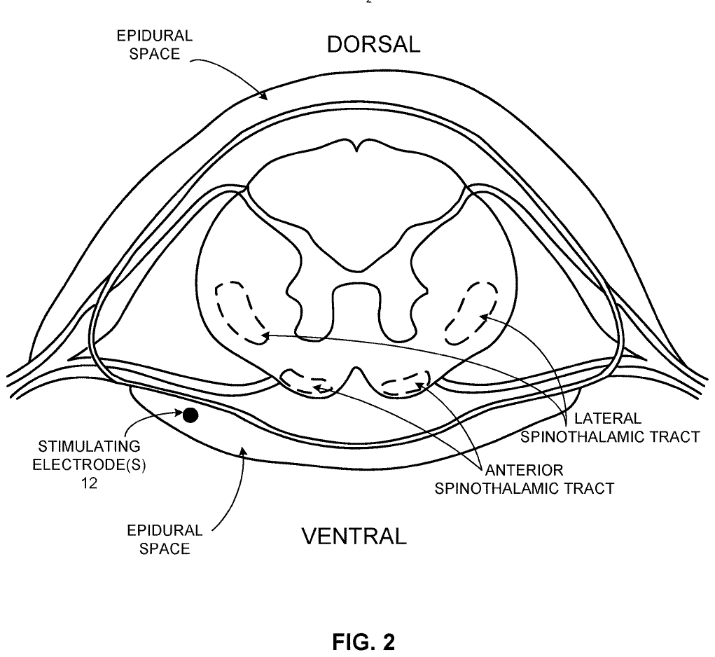

FIG. 2 shows a representative sketch of a thoracic cross section of the patient's spinal cord with the epidural space is shown (may not be drawn to scale or including all anatomical features). FIG. 2 shows an example placement of an electrode (represented as stimulating electrode(s) 12) at a location in the ventral epidural space, however this is merely an example. The electrode (represented as stimulating electrode(s) 12) can be positioned anywhere within the ventral epidural space and/or more than one electrode can be positioned anywhere within the ventral epidural space or on the ventral side of the spinal column, dependent on the pain to be at least partially relieved. Positioning stimulating electrode(s) 12 within the ventral space can bring the electrodes significantly closer to the anterior and lateral spinothalamic tracts than traditional dorsal positioning (such that the block can be a direct block). In some instances, the stimulating electrode(s) 12 can be placed on one or more portions the spinal column itself.

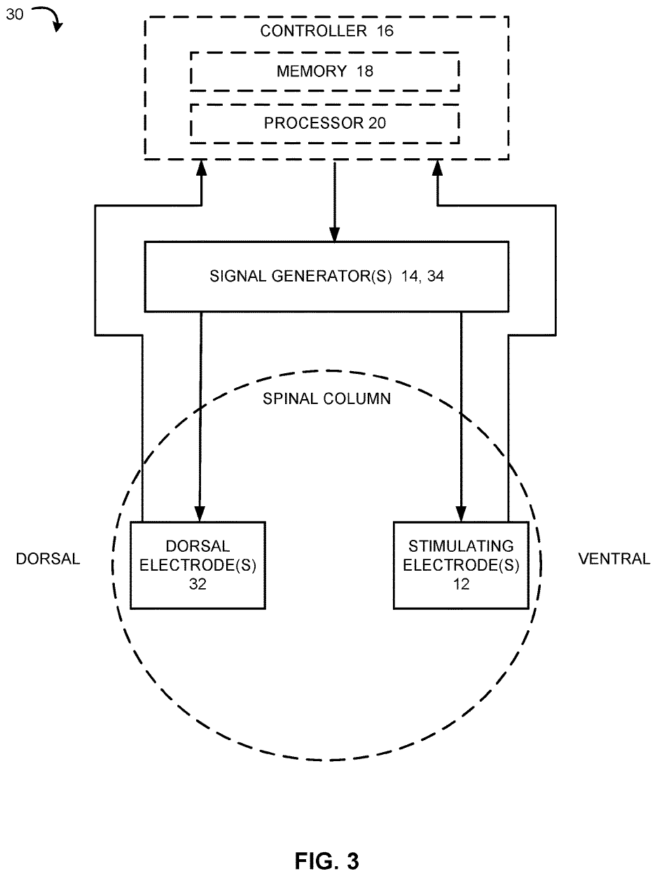

FIG. 3 shows another system 30 that governs the utilization of ventral SCS in combination with dorsal SCS. The ventral stimulating electrode(s) 12 can be at least one electrode implanted within the ventral epidural space or on the ventral side of the spinal column. The dorsal electrode(s) 32 can be another at least one electrode implanted within a dorsal column (e.g., the dorsal epidural space) of the spinal column of the subject. The system 30 can include at least one signal generator 14 connected to the at least one ventral SCS electrode (stimulating electrode(s) 12) and, optionally the dorsal electrode(s) 32, but can include a separate signal generator 34 for the dorsal electrodes. The signal generator(s) 14/34 can be connected to the controller 16 by at least one of a wired and/or wireless connection. The controller 16 can include non-transitory memory 18 configured to store instructions and processor 20 configured to execute the instructions. The controller 16 (via processor 20) can at least communicate information from the at least one electrode (stimulating electrode(s) 12) to the at least the other electrode (dorsal electrode(s) 32) and/or to each of the at least one electrode and the at least the other electrode. The information can, for instance, include an indication of a timing for delivering the stimulation and/or whether the at least one electrode and/or the other at least one electrode is delivering the stimulation at a given time.

### IV. Method

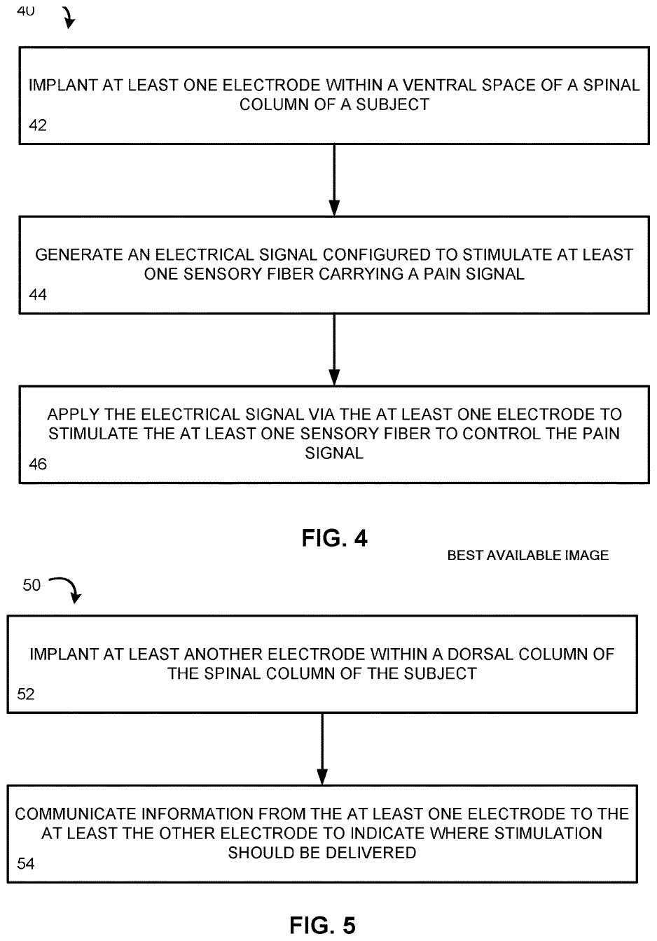

Another aspect of the present disclosure can include methods (FIGS. 4-6) that relate to at least partially relieving pain with ventral Spinal Cord Stimulation (SCS) that can be implemented with the systems 10 and 30 (shown in FIGS. 1 and 3). The methods can be executed by the system 10 of FIG. 1 and/or the system 30 of FIG. 3. The methods 40, 50, and 60 are illustrated as process flow diagrams with flowchart illustrations that can be implemented by one or more components of the systems 10 and/or 30. For purposes of simplicity, the methods 40, 50, and 60 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 40, 50, and 60.

FIG. 4 shows method 40 for at least partially relieving, controlling, and/or alleviating a pain of a subject using ventral SCS using the system 10 described with respect to FIG. 1. As a non-limiting example, the pain can be back pain, leg pain, neck pain, and/or neck pain. The pain can be caused, for example, by chronic pain syndrome, non-surgical back pain, complex regional pain syndrome, diabetic neuropathy, small fiber neuropathy, peripheral ischemia, and/or surgery. At 42, at least one electrode (e.g., stimulating electrode(s) 12) can be implanted within a ventral space (e.g., within the ventral epidural space or on the ventral side of the spinal column) of a spinal column of the subject. The at least one electrode can be implanted by any surgical means. The at least one electrode can be positioned near an anterior and/or a lateral spinothalamic tract of the spinal cord (e.g., also referred to as including pain fibers). If the at least one electrode is more than one, then each of the electrodes can be positioned near at least one of the anterior and/or the lateral spinothalamic tracts. For example, an electrode can be positioned in the ventrolateral epidural space as shown in FIG. 2. The at least one electrode can be implanted at any vertebrae or segment at any level of the spine (e.g., thoracic, lumber, sacral, cervical, etc.) dependent on the pain to be at least partially relieved. For example, the at least one electrode can be implanted on at least one thoracic level of the spine to at least partially relieve pain of the back and/or legs. In another example, the at least one electrode (stimulating electrode(s) 12) can be implanted on at least one cervical level of the spine to at least partially treat pain of the arms. In some instances, the at least one electrode (stimulating electrode(s) 12) can be implanted on a right side of the spine to relieve a pain on the right side of the body and/or on the left side of the spine to relieve a pain on the left side of the body.

At 44, an electrical signal can be generated by the signal generator (e.g., signal generator 14) to stimulate at least one sensory fiber carrying a pain signal. And at 46, the electrical signal can be applied via that at least one electrode to stimulate the at least one sensory fiber to control the pain signal. The at least one sensory fiber carrying the pain signal can be in the anterior and/or the lateral spinothalamic tract. The electrical signal can be generated with one or more parameters. The stimulation can be started or ended based on a user input. The one or more parameters of the electrical signal can be pre-programmed and/or selected by a controller 16. For example, the electrical signal can have a frequency between 0.001 MHz and 1 MHz and a current between 0.001 mA and 50 mA, or preferable between 0.001 mA and 5 mA. The electrical signal can be an alternating current or a direct current waveform, a monophasic or biphasic waveform. The electrical signal can be applied continuously or pulsed. For example, the electrical signal can have parameters of Fast-Acting Sub-perception Therapy stimulation (current: 1.2 mA, 230 μs, frequency: 90 Hz, and an active recharge phase). The electrical signal can be generated and applied for a time period ranging from 1 second to all day (e.g., 24 hours) to at least partially relieve the pain of felt by the patient. For example, the electrical signal can be applied to stimulate the at least one sensory fiber carrying the pain signal continuously for a time period. The time period can be started and/or stopped manually by the patient or can be a predetermined time period programmed into the system (e.g., system 10)

Method 50 shown in FIG. 5 can be used with method 40 in order to apply both ventral SCS and dorsal SCS to a subject to relieve, control, and/or alleviate the subject's pain using the system 30 of FIG. 3. At 52, at least another electrode (e.g., dorsal electrode(s) 32) can be implanted within a dorsal column of the spinal column of the subject (e.g., within the dorsal epidural space). The at least the other electrode (e.g., dorsal electrode(s)) can be implanted at the same time, prior to, or after the at least one electrode (e.g., ventral stimulating electrode(s)). The at least one (ventral) electrode and the at least the other (dorsal) electrode can be positioned at a same level of the spinal and/or at different levels of the spine. Both the at least one electrode (e.g., ventral stimulating electrode(s)) and the at least the other electrode (e.g., dorsal electrode(s)) can be connected to a system (e.g., controller, including a processor, and signal generator). At 54, information can be communicated, by the system, from the at least one electrode (e.g., ventral stimulating electrode(s)) to the at least the other electrode (e.g., dorsal electrode(s)), and vice versa, to indicate where the stimulation should be delivered. The information can also include, for instance, include an indication of a timing for delivering the stimulation and/or whether the at least one electrode and/or the other at least one electrode is delivering the stimulation at a given time. For example, stimulation can be alternated between dorsal and ventral electrodes, changed from one dorsal electrode to another or from one ventral electrode to another, and/or simultaneously applied through both dorsal and ventral electrode(s).

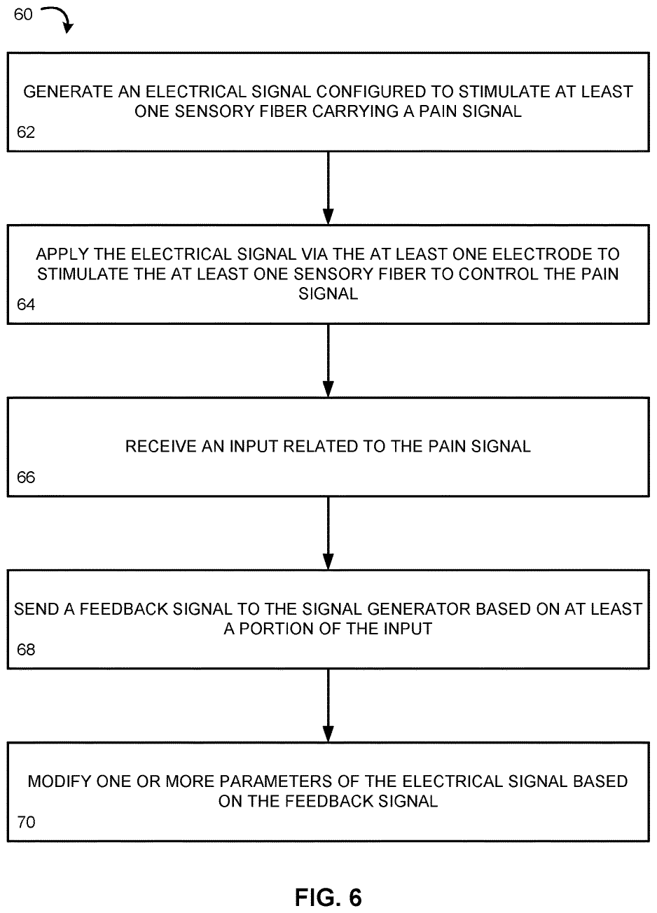

Method 60 of FIG. 6 shows a method for controlling ventral SCS with feedback using a system 10 including a controller having a non-transitory memory and a processor (e.g., controller 16, memory 18, and processor 20). Similar to the steps of method 40, at 62, an electrical signal is generated to stimulate the at least one sensory fiber carrying a pain signal and at 64, the electrical signal can be applied via the at least one electrode to stimulate the at least one sensory fiber to control the pain signal. At 66, an input related to the pain signal felt by the subject can be received. The input can be a manual input by the subject indicating an intensity and/or location of the pain and/or sensor recording, such as a recording of at least one ventral column evoked compound action potential indicating an intensity and/or location of the pain. When the input is a recording of at least one ventral column evoked compound action potential, then the method can also include implanting at least one sensing electrode (e.g., sensing electrode(s) 22) in and/or near the ventral column. At 68, a feedback signal can be sent to the signal generator (e.g., from the controller) based on at least a portion of the input related to the pain signal. The controller can determine the feedback signal based on at least a portion of the input related to the pain signal. For example, the controller may format the feedback signal based on the input signal at a time (e.g., manual input, increase or decrease stimulation effect, change stimulation location, or the like) and/or based on changes in the input signal for a period of time (e.g., input indicates pain is worsening or getting better of the period of time, pain location is changing over the period of time). At 70, one or more parameters of the electrical signal can be modified by the signal generator based on the feedback signal. The one or more parameters modified can include, but are not limited to, the frequency, the amplitude, the timing, and/or the location of application of the electrical signal.

### V. Experimental

Described herein is an investigation of the feasibility and potential efficacy of ventral column (VC) spinal cord stimulation (SCS) compared with traditional dorsal column (DC) SCS to treat chronic pain. As used herein, L refers to the lumbar region of the spine, T refers to the thoracic region of the spine, and S refers to the sacral region of the spine and the associated numbers refer to the traditional vertebrae and/or segments of each region.

### Methods

A 60-yr-old man was referred to an SCS trial after numerous surgical procedures. He underwent L3-4 and L4-5 bilateral laminectomy, followed by L3-4 and L4-5 redo laminectomy and L3 through L5 instrumented fusion. This was followed by L4-5 interbody graft migration and finally L4-5 revision arthrodesis. He had completed physical therapy with a home exercise program and continued to have significant difficulty with mobility, using a rolling walker, as well as difficulty with activities of daily living. He reported significant pain in his back and bilateral legs, with pain to light touch in dermatomes of bilateral L4, L5, and S1 vertebrae. The 60-yr-old man had absent bilateral Achilles reflexes and strength impairment with bilateral ankle dorsiflexion, extensor hallicus longus, and plantar flexion.

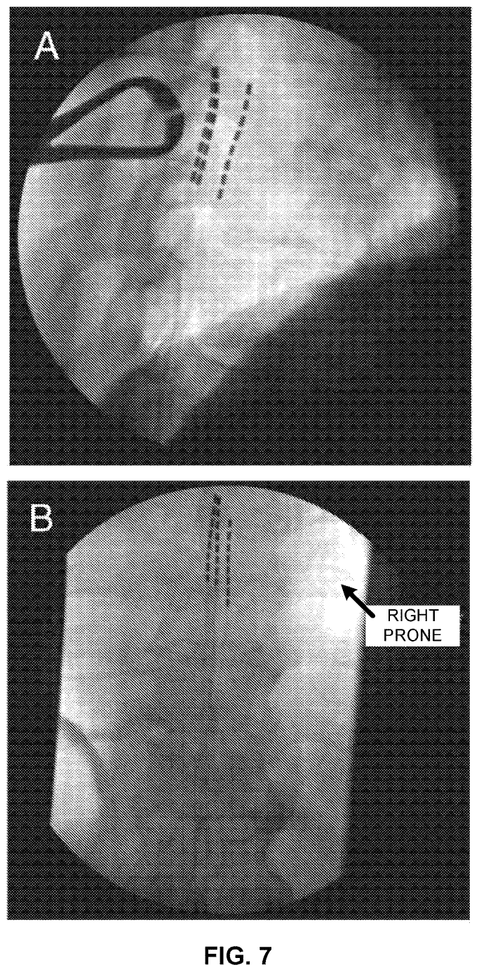

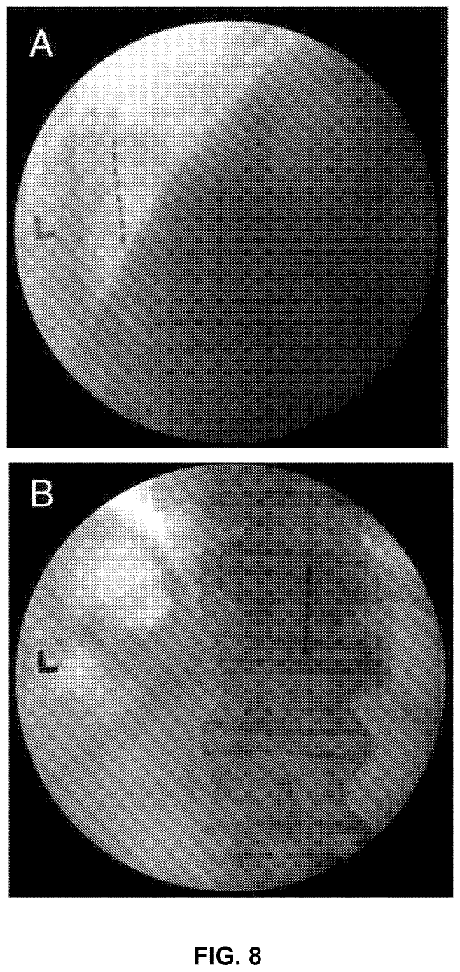

During the SCS trial, standard-of-care DC percutaneous leads were placed epidurally at left T12/L1 interspinous space, and lead tips advanced from the bilateral midline to the top of the T8 vertebral body end plate. Upon entry into the left L1/2 epidural space, the third lead was steered from left lateral entry point to the right, underneath the adjacent exiting nerve roots, and advanced. Lateral fluoroscopic view confirmed the location of third lead anterior to the spinal cord. Once confirmed, this lead was advanced to the top of the T8 vertebral body in the ventrolateral space (FIG. 7). FIG. 7, element A shows a sagittal view of the SCS leads. Two dorsal (dorsal column) leads (left two leads) and one ventral (ventrolateral column) lead (right head) were placed. FIG. 7, element B shows an anterior-posterior view of the SCS leads where the left two leads are the dorsal leads and the right lead is the ventral lead. Stimulation was applied using a Boston Scientific WaveWriter Alpha through Model SC-2218-50 Linear ST 8 contact lead. On trial days 1-7, DC SCS was optimized for back and leg pain relief as is the standard of care. For days 7-9, the DC leads were removed, and the right VC lead was repositioned to T11 based on paresthesia mapping covering low back and leg pain (FIG. 8). FIG. 8, element A shows a sagittal view of the ventral (ventrolateral) SCS lead positioned at T11 and FIG. 8, element B shows an anterior-posterior view of the ventral SCS lead. This study conformed to all Case Reports (CARE) guidelines and reports the required information accordingly. FIG. 3 shows a thoracic cross-section illustration of the placement of both the dorsal and ventral SCS leads with respect to the anterior and lateral spinothalamic tracts of the spinal cord.

### Results

For DC SCS, optimal pain relief was achieved with Fast-Acting Sub-perception Therapy (FAST) stimulation (2.4 mA, 220 μs, 90 Hz, active recharge) and Contour (2 mA, 200 μs, 130 Hz). For the right VC SCS, optimal pain relief was achieved with FAST (1.2 mA, 230 μs, 90 Hz, active recharge) stimulation.

TABLE 1
Pain ratings during course of SCS trial

| Scale | Category | Baseline | DC SCS | VC SCS |
|---|---|---|---|---|
| BPI-SF^{†} | Worse Pain | 10 | 4 | 3 |
| | Average Pain | 8 | 3 | 3 |
| | Current Pain | 6 | 4 | 3 |
| | Pain Interference | 8 | 7 | 5 |

SCS: Spinal Cord Stimulation, DC: Dorsal Column, VC: Ventral Column BPI-SF: Brief Pain Index Short Form
^{†}BPI scale range 0-10, with 0 indicating no symptoms and 10 the worst pain possible

The Brief Pain Index Short Form results are presented in Table 1. Scores for both DC SCS and VC SCS were better than before the trial, and VC SCS scores were comparable with DC SCS scores. The Numeric Rating Scale (NRS) scores for back and bilateral leg pain before and during DC SCS and VC SCS are presented in Table 2. The NRS scores for the week before the stimulation trial averaged 9 for bilateral leg pain and 8 for back pain. During DC SCS, bilateral leg and back pain NRS scores improved to 3. During right VC SCS, right leg and right back pain NRS scores improved to 3, whereas left leg and left back pain NRS scores were 9. When the patient received paresthesia-based stimulation for VC SCS, he reported no improvement in pain and, within 2 hours, requested to change back to nonparesthesia stimulation. Function improved with both DC SCS and VC SCS. Finally, with VC SCS, the patient did not perceive movement-evoked electrical shocks that he felt, and that often occur, with DC SCS.

TABLE 2
Numeric Rating Scale of SCS trial

| | NRS |
|---|---|
| Bilateral Leg Pain Week Before SCS Trial | 8 |
| Low Back Pain Week Before SCS Trial | 8 |
| Bilateral Leg Pain with Bilateral Dorsal Column SCS | 3 |
| Back Pain with Dorsal Bilateral Column SCS | 3 |
| Right Leg Pain with Right Ventral Column SCS | 3 |
| Right Back Pain with Right Ventral Column SCS | 3 |
| Left Leg Pain with Right Ventral Column SCS | 9 |
| Left Back Pain with Right Ventral Column SCS | 9 |

Accordingly, VC SCS provided a reasonable outcome in a patient with postlumbar laminectomy syndrome and chronic radicular pain. The VC SCS provided back and leg coverage when the lead was placed at vertebral body T11, significantly more distal than DC SCS. Right anterolateral VC SCS at this level resulted in right leg pain relief, contrary to expected contralateral sensory decussation. Right cord stimulation resulted in right back and right pain relief, and no left back and leg relief, suggesting that therapeutic VC SCS does not propagate through cerebral spinal fluid, rather it acts through direct spinal activation.

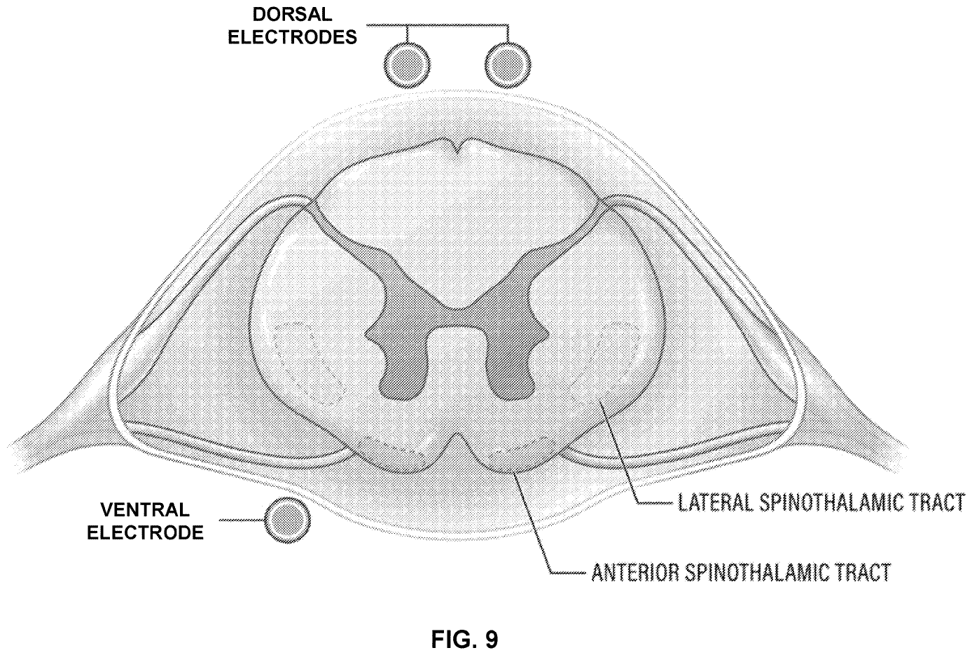

It should be noted that FIG. 9 shows the ventral electrode implanted on the ventral side of the spinal cord and the dorsal electrodes implanted on the dorsal side of the spinal cord. The ventral electrode was implanted on the left side (as viewed from the cross-section) of the spinal column near a lateral spinothalamic tract and an anterior spinothalamic tract (with the pain fibers) enabling direct stimulation.

From the above description, those skilled in the art will perceive improvements, changes and modifications. Such improvements, changes and modifications are within the skill of one in the art and are intended to be covered by the appended claims. All patents, patent applications, and publications cited herein are incorporated by reference in their entirety.

What is claimed is:

1. A method comprising:
implanting at least one electrode within a ventral space of a spinal column of a subject, wherein the at least one electrode is positioned near an anterior and/or a lateral spinothalamic tract;
generating, by a signal generator, an electrical signal configured to stimulate at least one sensory fiber carrying a pain signal in the anterior and/or the lateral spinothalamic tract; and
applying the electrical signal via the at least one electrode to the anterior and/or the lateral spinothalamic tract to stimulate the at least one sensory fiber to override the pain signal with non-painful stimulus induced activity to at least partially relieve pain felt by the subject.

2. The method of claim 1, wherein the implanting further comprises implanting the at least one electrode on at least one cervical level, at least one thoracic level, and/or at least one lumbar level of the spine.

3. The method of claim 2, wherein the implanting further comprises implanting the at least one electrode at least one thoracic level of the spine and the pain at least partially relieved comprises a pain of the back and/or the legs of the subject.

4. The method of claim 1, further comprising beginning and/or ending the stimulation after receiving a user input.

5. The method of claim 1, wherein the pain is chronic pain.

6. The method of claim 1, wherein the pain is back pain, leg pain, neck pain, and/or arm pain.

7. The method of claim 1, wherein the pain is caused by at least one of complex regional pain syndrome, diabetic neuropathy, small fiber neuropathy, peripheral ischemia, and/or surgery.

8. The method of claim 1, wherein the applying further comprises stimulating the at least one sensory fiber carrying the pain signal continuously for a time period.

9. The method of claim 1, further comprising:
receiving an input related to the pain being treated;
sending a feedback signal to the signal generator based on at least a portion of the input; and
modifying one or more parameters of the electrical signal based on the feedback signal.

10. The method of claim 9, wherein the input is a manual indication of pain or a recording of at least one ventral column evoked compound action potential.

11. The method of claim 1, further comprising:
implanting at least another electrode within a dorsal column of the spinal column of the subject; and
communicating, by a system comprising a processor, information from the at least one electrode to the at least the other electrode to indicate where the stimulation should be delivered.

12. The method of claim 1, wherein the electrical signal comprises at least one of a frequency between 0.001 and 1 MHz and a current between 0.001 mA and 50 mA.

13. A system comprising:
a signal generator configured to generate an electrical signal, wherein the electrical signal is configured to stimulate at least one sensory fiber carrying a pain signal in an anterior and/or a lateral spinothalamic tract;
at least one electrode configured to be implanted within a ventral space of the spinal column of the subject near the anterior and/or the lateral spinothalamic tract and to apply the electrical signal to the at least one sensory fiber to stimulate the at least one sensory fiber to override the pain signal with non-painful stimulus induced activity to at least partially relieve pain felt by the subject.

14. The system of claim 13, further comprising:
at least one sensing electrode configured to record one or more evoked compound action potentials related to the pain of the subject; and
a controller, comprising a non-transitory memory and a processor, configured to:
receive a recording of the one or more evoked compound action potentials;
modulate at least one parameter of the electrical signal in response to the one or more evoked compound action potentials to improve pain management; and
send the modulated at least one parameter to the signal generator.

15. The system of claim 13, further comprising:
another at least one electrode configured to be implanted within a dorsal column of the spinal column of the subject; and
another system comprising a processor configured to:
communicate information from the at least one electrode to the at least the other electrode, wherein the information indicates a timing for delivering the stimulation and/or whether the at least one electrode and/or the other at least one electrode is delivering the stimulation.

16. The system of claim 13, wherein the at least one electrode comprises a plurality of contacts.

17. The system of claim 13, wherein the at least one electrode comprises at least one percutaneously implanted cylindrical electrode or at least one implantable paddle electrode.

18. The system of claim 13, wherein the at least one electrode comprises platinum-iridium.

19. The system of claim 13, wherein the electrical signal comprises a frequency between 0.001 and 1 MHz and/or a current between 0.001 mA and 50 mA.

20. The system of claim 13, wherein the at least one electrode is configured to be implanted on at least one thoracic level of the spine and the pain at least partially relieved comprises a pain of the back and/or legs or on at least one cervical level and the pain at least partially relieved comprises a pain of the arms.
